(19) United States
(12) Patent Application Publication
(10) Pub. No.: US 2024/0423510 A1
(43) Pub. Date: Dec. 26, 2024

(54) MINIATURIZED BLOOD GLUCOSE MEASUREMENT MODULE BY USING POLARIZED LIGHT

(71) Applicant: Taiwan-Asia Semiconductor Corporation, Hsinchu City (TW)

(72) Inventors: Tzu-Hui LIN, Hsinchu City (TW); Sheng-Wei CHEN, Hsinchu City (TW)

(21) Appl. No.: 18/642,345

(22) Filed: Apr. 22, 2024

(30) Foreign Application Priority Data
Jun. 20, 2023 (TW) 112123143

(51) Int. Cl. A61B 5/145; A61B 5/1455

(52) U.S. Cl. CPC A61B 5/14532; A61B 5/14558; A61B 2560/0462; A61B 2562/0233; A61B 2562/185

(57) ABSTRACT

A miniaturized blood glucose measurement module using polarized light is provided, which includes an electromagnet assembly, a light-emitting assembly, and a light-receiving assembly. The electromagnet assembly includes a metal base and a coil. The metal base includes an accommodating portion and a side surface. The accommodating portion is located within a scope of the side surface, and the coil is wound on the side surface. The light-emitting assembly is embedded in the accommodating portion and includes a light-emitting element and a first polarizing element. A light emitted by the light-emitting element passes through the first polarizing element to generate a polarized light, and the light-emitting assembly forms a light-emitting surface. The light-receiving assembly is embedded in the accommodating portion and adjacent to the light-emitting assembly, and the light-receiving assembly includes a light-sensing element, a magnetic crystal and a second polarizing element. The light-sensing element receives the reflected polarized light which passes through the magnetic crystal and the second polarizing element sequentially, and the light-receiving assembly forms a light-receiving surface.

### CROSS-REFERENCES TO RELATED APPLICATIONS

This application claims the benefit of priority to Taiwanese Patent Application No. 112123143 filed on Jun. 20, 2023, which is hereby incorporated by reference in its entirety.

### BACKGROUND OF THE INVENTION

### Field of the Invention

The present invention relates to a blood glucose measurement module, in particular to a miniaturized blood glucose measurement module by using polarized light.

### Descriptions of the Related Art

In recent years, light sources that can emit polarized light have been used in non-invasive blood glucose detection modules. For example, Publication No. CN1676100A discloses a blood glucose detector. Since the glucose in blood sugar has optical rotation characteristics, a single polarized light source is applied to rotate the polarization of the light source by an angle after passing through human tissue. Then, a Faraday rotator is applied, by using the Faraday effect, the light source is rotated at another specific angle, and finally, the penetration intensity of the light source at different angles is received and analyzed through the photosensitive element to obtain the blood glucose concentration of the specimen. The Faraday rotator includes magnets and magnetic crystals. However, for the Faraday rotator to provide the effect of rotating the angle of the light source, the conventional blood glucose detection module using polarized light detection technology must be equipped with a coil structure that can generate an induced magnetic field to surround the outside of the Faraday rotator. The coil structure of this design makes the system bulky, causing the entire blood glucose detection module unable to meet the requirements for miniaturization and difficult to be applied to wearable devices.

Therefore, how to design a blood glucose measurement module that can improve the aforementioned problems is indeed a subject worthy of study.

### SUMMARY OF THE INVENTION

The main objective of the present invention is to provide a miniature blood glucose measurement module combining an electromagnet assembly and polarized light.

To achieve the above objective, the miniaturized blood glucose measurement module by using polarized light of the present invention comprises an electromagnet assembly, a light emitting assembly, and a light receiving assembly. The electromagnet assembly includes a metal base and a coil, the metal base includes an accommodating portion and a side surface, wherein the accommodating portion is located within a scope of the side surface, and the coil is wound on the side surface. The light-emitting assembly is embedded in the accommodating portion and includes a light-emitting element and a first polarizing element, wherein a light emitted by the light-emitting element passes through the first polarizing element to generate a polarized light, and the light-emitting assembly forms a light-emitting surface. The light-receiving assembly is embedded in the accommodating portion and includes a light-sensing element, a magnetic crystal, and a second polarizing element; wherein the light-sensing element receives the reflected polarized light which passes through the magnetic crystal and the second polarizing element sequentially, and the light receiving assembly forms a light-receiving surface.

In one embodiment of the present invention, the metal base further includes a first surface and a second surface, which are opposing to each other, the side surface is located between the first surface and the second surface, and the light-emitting surface of the light-emitting assembly, the light-receiving surface of the light-receiving assembly and the first surface are located on the same plane.

In one embodiment of the present invention, the accommodating portion is a groove recessed from the first surface toward the second surface, and a depth of the groove is less than a height of the metal base.

In one embodiment of the present invention, the accommodating portion is a through hole penetrating from the first surface to the second surface.

In one embodiment of the present invention, the metal base is made of soft magnetic material or hard magnetic material.

In one embodiment of the present invention, a wavelength range corresponding to the light-emitting assembly is between 500 nm and 1800 nm.

In one embodiment of the present invention, the light-emitting assembly is a vertical resonant cavity surface-emitting laser or a light-emitting diode.

In one embodiment of the present invention, the magnetic crystal is made of a material that can produce magnetization under the influence of an external magnetic field.

In one embodiment of the present invention, the light-emitting assembly further includes a first cover, and the first cover is disposed on a side of the first polarizing element facing away from the light-emitting element; the light-receiving assembly further includes a second cover, and the second cover is disposed on a side of the magnetic crystal facing away from the light sensing element.

In one embodiment of the present invention, the first cover and the second cover are made of glass or plastic material.

In one embodiment of the present invention, the light-emitting assembly further includes a first substrate and a first light-shielding structure, the light-emitting element and the first light-shielding structure are disposed on the first substrate, and the first light-shielding structure is located around the light-emitting element; the light-receiving assembly further includes a second substrate and a second light-shielding structure, the light sensing element and the second light-shielding structure are arranged on the second substrate, and the second light-shielding structure is located around the light sensing element.

In one embodiment of the present invention, the first light-shielding structure and the second light-shielding structure are made of non-light-transmitting epoxy resin.

Accordingly, the miniature blood glucose detection module of the present invention can directly drive the magnetic crystal of the light-receiving assembly to change the polarization angle of the light through the electromagnet assembly. Compared with the conventional blood glucose detection module using polarized, the size of the module can be effectively reduced so that the assembly is miniaturized, and the electromagnet assembly can significantly improve the Faraday effect on the magnetic crystal compared to the Faraday rotator, and enhance the difference between optical signals to improve the accuracy of blood glucose detection.

### BRIEF DESCRIPTION OF THE DRAWINGS

FIG. 1 is a schematic diagram of the miniaturized blood glucose measurement module using polarized light of the first embodiment of the present invention.

FIG. 2 is a top view of the miniaturized blood glucose measurement module using polarized light of the first embodiment of the present invention.

FIG. 3 is a schematic diagram of the miniaturized blood glucose measurement module using polarized light of the second embodiment of the present invention.

### DETAILED DESCRIPTION OF THE PREFERRED EMBODIMENT

Since various modifications and embodiments are only illustrative and not limiting, after reading this specification, those with ordinary skill in the art may conceive of other variations and embodiments that do not depart from the scope of the present invention. The features and advantages of such embodiments will be further highlighted based on the detailed description and the scope of the claims set forth below.

In this document, the terms “one” or “a” are used to describe the components and elements disclosed herein. This is done for convenience and to provide a general meaning to the scope of the present invention. Therefore, unless otherwise explicitly indicated, such descriptions should be understood to encompass one or at least one, and the singular also includes the plural.

In this document, terms like “first” or “second” and similar ordinal numbers are primarily used to distinguish or refer to similar or analogous components or structures and do not necessarily imply an order in space or time. It should be understood that in certain situations or configurations, ordinal numbers can be used interchangeably without affecting the implementation of the present disclosure.

In this document, terms like “comprising,” “including,” “having,” or any similar expressions are intended to cover non-exclusively inclusive entities. For example, components or structures containing multiple elements are not limited solely to the listed elements in this document but may include other elements that are typically inherent to the component or structure even if not explicitly listed.

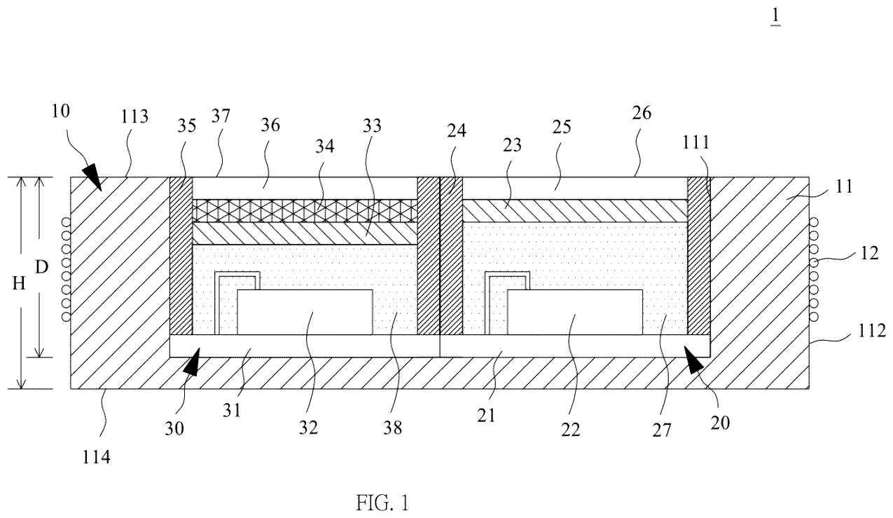

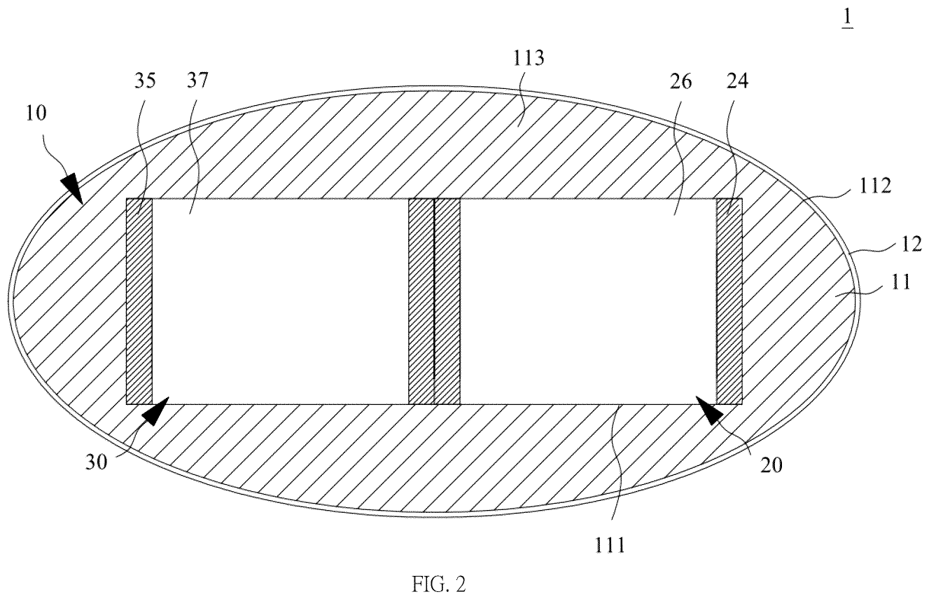

Please refer to FIG. 1 and FIG. 2, FIG. 1 is a schematic diagram of the first embodiment of the miniature blood glucose measurement module of the present invention, and FIG. 2 is a top view of the first embodiment of the miniature glucose measurement module of the present invention. As shown in FIG. 1 and FIG. 2, the miniature blood glucose measurement module 1 of the present invention includes an electromagnet assembly 10, a light-emitting assembly 20 and a light-receiving assembly 30, wherein the light-emitting assembly 20 and the light-receiving assembly 30 are arranged on the electromagnet assembly 10. The electromagnet assembly 10 mainly generates an induced magnetic field through the current supply. The light-emitting assembly 20 mainly generates polarized light to illuminate the skin. The light-receiving assembly 30 mainly receives the polarized light that is diffusely reflected by the skin and then transmitted back.

The electromagnet assembly 10 includes a metal base 11 and a coil 12. The metal base 11 serves as the basic structural member of the electromagnet assembly 10, and the metal base 11 has a three-dimensional block structure. In terms of appearance design, the lateral outer contour of the metal base 11 can be circular, oval, rectangular, or other shapes. For example, in this embodiment, it is oval, but the invention is not limited thereto. In the present invention, the metal base 11 can be made of soft magnetic materials (such as iron, iron-cobalt alloy, iron-silicon steel, iron-nickel alloy, etc.) or hard magnetic materials (such as ferrite, neodymium iron boron, etc.) according to different needs. The metal base 11 includes an accommodating portion 111, a side surface 112, an opposite first surface 113, and a second surface 114. The first surface 113 and the second surface 114 are two planes parallel to each other, and the side surface 112 is located between the first surface 113 and the second surface 114. The accommodating portion 111 forms an accommodating space for disposing the light-emitting assembly 20 and the light-receiving assembly 30, and the accommodating portion 111 is located within a range surrounded by the side surface 112 (or the lateral outer contour of the metal base 11). That is to say, a distance is maintained between the four sides of the accommodating portion 111 and the side surface 112. In this embodiment, the accommodating portion 111 is a groove recessed from the first surface 113 toward the second surface 114, and the depth D of the groove is smaller than the height H of the metal base 11. That is to say, a distance is maintained between the bottom surface of the groove-shaped accommodating portion 111 and the second surface 114.

The coil 12 is wound around the side surface 112 of the metal base 11. The total number of winding turns of the coil 12 on the side surface 112 is between 1 and 300. As the total number of winding turns of the coil 12 is different, the magnitude of the current flowing into the coil will be adjusted accordingly. Generally, the current will be between 1˜100 mA (milliamp). In this embodiment, the total number of winding turns of the coil 12 is about 50, but the invention is not limited thereto. In addition, the coil 12 can be metal wires of different wire diameters according to the design. For example, an enameled wire with a wire diameter of AWG38 (equivalent to a wire diameter of 0.102 mm) can be used, or an enameled wire with a wire diameter of AWG33˜46 (equivalent to a wire diameter of 0.18 mm˜0.041 mm) can be used. A current of 15 mA is passed through the coil to generate a corresponding induced magnetic field, but the invention is not limited thereto.

The light-emitting assembly 20 is embedded in the accommodating portion 111 of the metal base 11, and the light-emitting assembly 20 includes a first substrate 21, a light-emitting element 22, and a first polarizing element 23. The first substrate 21 serves as the basic structural component of the light-emitting assembly 20, and the first substrate 21 can be used to carry the light-emitting element 22, the first polarizing element 23, and other related components. The light-emitting element 22 is disposed on the first substrate 21 and serves as a light source to emit light. The light-emitting element 22 can be electrically connected to the first substrate 21 through metal wires. In the present invention, the light-emitting element 22 is a laser diode, such as a vertical cavity surface emitting laser (VCSEL), but the present invention is not limited thereto. For example, the light-emitting element 22 may also be a light-emitting diode (LED) or other types of light sources. The wavelength range corresponding to the light-emitting element 22 is approximately between 500 nm and 1800 nm. For example, in one embodiment of the present invention, the wavelength corresponding to the light-emitting element 22 may be 650 nm. The first polarizing element 23 is disposed on the emission path of the light emitted by the light-emitting element 22, such as directly above the light-emitting element 22, so that the light passes through the first polarizing element 23 to generate linearly polarized light. In the present invention, the first polarizing element 23 is made of polymer or metal material, but the present invention is not limited thereto.

In one embodiment of the present invention, the light-emitting assembly 20 further includes a first light-shielding structure 24. The first light-shielding structure 24 is disposed on the first substrate 21 and is located around the light-emitting element 22. The first light-shielding structure 24 is mainly used to block the side scattering of light emitted by the light-emitting element 22 so that the light can be concentrated and emitted toward the first polarizing element 23. The first light-shielding structure 24 is made of non-transparent epoxy resin, such as encapsulating vinyl, but the invention is not limited thereto.

In one embodiment of the present invention, the light-emitting assembly 20 further includes a first cover 25. The first cover 25 is stacked on the first polarizing element 23, and the side of the first cover 25 facing away from the first polarizing element 23 (that is, the side facing the skin) forms the light-emitting surface 26 of the light-emitting assembly 20. The first cover 25 serves as a protective member of the light-emitting assembly 20, and the first cover 25 is made of light-transmissive glass or plastic material, but the invention is not limited thereto.

In addition, in the light-emitting assembly 20, packaging materials can be further filled between the first substrate 21, the first polarizing element 23, and the first light-shielding structure 24 to form the first packaging structure 27. The first packaging structure 27 is used to package and fix the light-emitting element 22 and related metal wires. The first packaging structure 27 is made of light-transmissive epoxy resin, but the invention is not limited thereto.

In terms of design, the light-emitting assembly 20 is positioned after being embedded in the accommodating portion 111 of the metal base 11, and the light-emitting surface 26 of the light-emitting assembly 20 and the first surface 113 of the metal base 11 are coplanar, thereby maintaining smoothness and consistency of the appearance surface of the overall structure.

The light-receiving assembly 30 includes a second substrate 31, a light-sensing element 32, a second polarizing element 33, and a magnetic crystal 34. The second substrate 31 serves as the basic structural component of the light-receiving assembly 30, and the second substrate 31 can be used to carry the light-sensing element 32, the second polarizing element 33, the magnetic crystal 34, and other related components. The light-sensing element 32 is disposed on the second substrate 31, and the light-sensing element 32 is used to receive reflected light for subsequent detection. The light-sensing element 32 can be electrically connected to the second substrate 31 through metal wires. In the present invention, the light-sensing element 32 is a photodetector, but the present invention is not limited thereto. The second polarizing element 33 is disposed on the incident path of the reflected light received by the light-sensing element 32, for example, directly above the light-sensing element 32. In the present embodiment, the second polarizing element 33 is made of polymer or metal material, but the present invention is not limited thereto. The magnetic crystal 34 is stacked on the second polarizing element 33 and is also located on the incident path of the reflected light received by the light-sensing element 32, such as directly above the light-sensing element 32 so that the reflected light passes through the magnetic crystal 34. The second polarizing element 33 is then received by the light-sensing element 32. The magnetic crystal 34 is mainly used to change the polarization angle of the reflected light, and the magnetic crystal 34 can rotate the crystal through an external magnetic field. Therefore, the magnetic crystal 34 is made of a material that can produce a magnetization phenomenon under the influence of an external magnetic field. In the present invention, the magnetic crystal 34 uses Magneto-Optic Faraday Rotator Garnet Crystals as a rotating crystal that reflects the polarization angle of the reflected light. However, the magnetic crystal 34 can also use other devices that can apply variable magnetic fields or a crystal that is controlled by an electric field to rotate the polarization angle of the incident light, such as a liquid crystal.

In one embodiment of the present invention, the light-receiving assembly 30 further includes a second light-shielding structure 35. The second light-shielding structure 35 is provided on the second substrate 31 and is located around the light-sensing element 32. The second light-shielding structure 35 is mainly used to block the reflected light that sequentially passes through the magnetic crystal 34 and the second polarizing element 33 from being scattered laterally, so that the reflected light can be concentrated and received by the light-sensing element 32; and to prevent ambient light from escaping from the side. The light-sensing element 32 is directed toward the incident light. The second light-shielding structure 35 is made of non-transparent epoxy resin, such as encapsulating vinyl, but the invention is not limited thereto.

In one embodiment of the present invention, the light-receiving assembly 30 further includes a second cover 36. The second cover 36 is stacked on the magnetic crystal 34, and the side of the second cover 36 facing away from the magnetic crystal 34 (that is, the side facing the skin) forms the light-receiving surface 37 of the light-receiving assembly 30. The second cover 36 serves as a protective member of the light-receiving assembly 30, and the second cover 36 is made of light-transmissive glass or plastic material, but the invention is not limited thereto.

In addition, in the light-receiving assembly 30, packaging materials can be further filled between the second substrate 31, the second polarizing element 33, and the second light shielding structure 35 to form the second packaging structure 38. The second packaging structure 38 is used to package and fix the light-sensing element 32 and related metal wires. The second packaging structure 38 is made of light-transmissive epoxy resin, but the invention is not limited thereto.

In terms of design, the light-receiving assembly 30 is positioned after being embedded in the accommodating portion 111 of the metal base 11, and the light-receiving surface 37 of the light-receiving assembly 30 and the first surface 113 of the metal base 11 are coplanar, thereby maintaining the smoothness and consistency of the appearance surface of the overall structure. That is to say, the light-emitting surface 26 of the light-emitting assembly 20, the light-receiving surface 37 of the light-receiving assembly 30, and the first surface 113 of the metal base 11 are located on the same plane.

In the actual practice of the miniaturized blood glucose measurement module using polarized light, the micro-polarized blood glucose measurement module 1 of the present invention first emits light through the light-emitting element 22 of the light-emitting assembly 20. The light will sequentially pass through the first polarizing element 23 and the first cover 25 and generate polarization. After the light arrives, it can perform corresponding blood sugar detection based on the glucose in human tissue. After that, the light reflected from the glucose in the skin will sequentially pass through the second cover 36 of the light-receiving assembly 30, the magnetic crystal 34, and the second polarizing element 33, and then be received by the light-sensing element 32 to perform subsequent blood glucose detection signals through Analysis and processing. Among them, by passing a current through the coil 12 of the electromagnet assembly 10, the electromagnet assembly 10 generates an induced magnetic field, which in turn affects the magnetic crystal 34 to generate crystal rotation to change the angle of the polarization direction of the light source.

To achieve a miniaturized size design, in this embodiment, it is assumed that the length and width of the light-emitting assembly 20 and the light-receiving assembly 30 are both x mm, and the height is h mm. The metal base 11 with an oval lateral outer contour has a corresponding long axis of 3x mm and a short axis between 2x mm and 3x mm. The height H of the metal base 11 is not less than h mm. The length of the accommodating portion 111 of the metal base 11 is x+0.1 mm, the width is 2x+0.1 mm, and the height is h+0.1 mm. For example, when x is 5 mm, the overall volume of the miniature blood glucose measurement module 1 of the present invention can be miniaturized, which is beneficial for use in wearable devices and can target the skin surface of any part of the body to perform a blood glucose test.

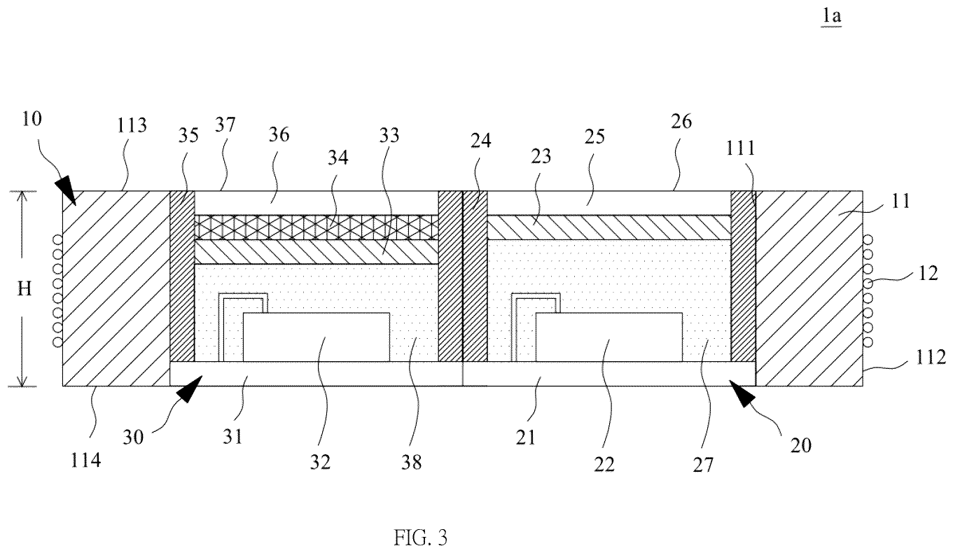

Please refer to FIG. 3 which is a schematic diagram of the second embodiment of the miniature blood glucose measurement module of the present invention. This embodiment is a modification of the aforementioned first embodiment, and mainly makes structural changes to the accommodating portion of the metal base. As shown in FIG. 3, in this embodiment, the accommodating portion 111 of the metal base 11 of the electromagnet assembly 10 of the miniature blood glucose measurement module 1a penetrates from the first surface 113 to the second surface 114 perforation. In terms of design, the height H of the metal base 11 is equal to the height of the light-emitting assembly 20 and the light-receiving assembly 30. When the light-emitting assembly 20 and the light-receiving assembly are embedded in the accommodating portion 111 of the metal base 11, they are positioned. At this time, the light-emitting surface 26 of the light-emitting assembly 20, the light-receiving surface 37 of the light-receiving assembly 30 and the first side 113 are coplanar, and the bottom surface of the first substrate 21 of the light-emitting assembly 20, the bottom surface of the second substrate 31 of the light-receiving assembly 30, and the second surface 114 of the metal base 11 are coplanar, thereby not only maintaining the appearance of the overall structure. In terms of flatness and consistency, compared with the aforementioned first embodiment, the height H of the metal base 11 is reduced to further meet the thinning requirement and improve the flexibility of module installation.

The above embodiments are essentially provided for auxiliary explanation, and are not intended to limit the embodiments of the claimed subject matter or their applications or uses. Furthermore, even though at least one illustrative embodiment has been presented in the foregoing embodiments, it should be understood that there can still be numerous variations within the scope of the invention. It should also be understood that the embodiments described herein are not intended to limit the scope, application, or configuration of the claimed subject matter in any way. On the contrary, the foregoing embodiments will provide a convenient guide for those skilled in the art to implement one or more embodiments of the claimed subject matter. Moreover, various changes in the functionality and arrangement of components can be made within the scope defined by the claims, and the claims encompass known equivalents and foreseeable equivalents at the time of filing this patent application.

What is claimed is:

1. A miniaturized blood glucose measurement module using polarized light, comprising:
an electromagnet assembly including a metal base and a coil, the metal base includes an accommodating portion and a side surface, wherein the accommodating portion is located within a scope of the side surface, and the coil is wound on the side surface;
a light-emitting assembly being embedded in the accommodating portion and includes a light-emitting element and a first polarizing element, wherein a light emitted by the light emitting element passes through the first polarizing element to generate a polarized light, and the light emitting assembly forms a light-emitting surface; and
a light-receiving assembly including a light-sensing element, a magnetic crystal, and a second polarizing element; wherein the light-sensing element receives the reflected polarized light which passes through the magnetic crystal and the second polarizing element sequentially, and the light-receiving assembly forms a light-receiving surface.

2. The miniaturized blood glucose measurement module using polarized light claimed in claim 1, wherein the metal base further includes a first surface and a second surface, which are opposing to each other, the side surface is located between the first surface and the second surface, and the light-emitting surface of the light-emitting assembly, the light-receiving surface of the light-receiving assembly and the first surface are located on the same plane.

3. The miniaturized blood glucose measurement module using polarized light claimed in claim 2, wherein the accommodating portion is a groove recessed from the first surface toward the second surface, and a depth of the groove is less than a height of the metal base.

4. The miniaturized blood glucose measurement module using polarized light claimed in claim 2, wherein the accommodating portion is a through hole penetrating from the first surface to the second surface.

5. The miniaturized blood glucose measurement module using polarized light claimed in claim 1, wherein the metal base is made of soft magnetic material or hard magnetic material.

6. The miniaturized blood glucose measurement module using polarized light claimed in claim 1, wherein a wavelength range corresponding to the light-emitting assembly is between 500 nm and 1800 nm.

7. The miniaturized blood glucose measurement module using polarized light claimed in claim 6, wherein the light-emitting assembly is a vertical resonant cavity surface-emitting laser or a light-emitting diode.

8. The miniaturized blood glucose measurement module using polarized light claimed in claim 1, wherein the magnetic crystal is made of a material that can produce magnetization under the influence of an external magnetic field.

9. The miniaturized blood glucose measurement module using polarized light claimed in claim 1, wherein the light-emitting assembly further includes a first cover, and the first cover is disposed on a side of the first polarizing element facing away from the light-emitting element; the light-receiving assembly further includes a second cover, and the second cover is disposed on a side of the magnetic crystal facing away from the light sensing element.

10. The miniaturized blood glucose measurement module using polarized light claimed in claim 9, wherein the first cover and the second cover are made of glass or plastic material.

11. The miniaturized blood glucose measurement module using polarized light claimed in claim 1, wherein the light-emitting assembly further includes a first substrate and a first light-shielding structure, the light-emitting element and the first light-shielding structure are disposed on the first substrate, and the first light-shielding structure is located around the light-emitting element; the light-receiving assembly further includes a second substrate and a second light-shielding structure, the light sensing element and the second light-shielding structure are arranged on the second substrate, and the second light-shielding structure is located around the light sensing element.

12. The miniaturized blood glucose measurement module using polarized light claimed in claim 11, wherein the first light-shielding structure and the second light-shielding structure are made of non-light-transmitting epoxy resin.
